(19) United States
(12) Patent Application Publication
(10) Pub. No.: US 2016/0054901 A1
(43) Pub. Date: Feb. 25, 2016

(54) METHOD, APPARATUS, AND SYSTEM FOR OUTPUTTING MEDICAL IMAGE REPRESENTING OBJECT AND KEYBOARD IMAGE

(71) Applicant: SAMSUNG MEDISON CO., LTD., Hongcheon-gun (KR)

(72) Inventors: Sun-mo YANG, Hongcheon-gun (KR); Seung-ju LEE, Hongcheon-gun (KR)

(73) Assignee: SAMSUNG MEDISON CO., LTD., Hongcheon-gun (KR)

(21) Appl. No.: 14/712,301

(22) Filed: May 14, 2015

Related U.S. Application Data
(60) Provisional application No. 62/040,644, filed on Aug. 22, 2014.

(30) Foreign Application Priority Data
Dec. 4, 2014 (KR) 10-2014-0173244

(51) Int. Cl. G06F 3/0484; A61B 8/00; G06F 19/00; G06T 3/40; G06F 3/0488; A61B 8/14; A61B 8/08

(52) U.S. Cl. CPC G06F 3/04845; A61B 8/14; A61B 8/464; A61B 8/5207; G06T 3/40; G06F 3/04886; G06F 3/04842; G06F 19/321

(57) ABSTRACT

Disclosed is a method of outputting a medical image representing an object and a keyboard image. The method includes displaying the medical image and the keyboard image in different regions of a single screen, performing image processing on the medical image, based on a first user input which is input via the keyboard image, and displaying a result of the image processing on the single screen.

### RELATED APPLICATIONS

This application claims the benefit of U.S. Provisional Application No. 62/040,644, filed on Aug. 22, 2014, in the US Patent Office and Korean Patent Application No. 10-2014-0173244, filed on Dec. 4, 2014, in the Korean Intellectual Property Office, the disclosures of which are incorporated herein in their entireties by reference.

### BACKGROUND

1. Field

One or more exemplary embodiments relate to a method, apparatus, and system for outputting a medical image representing an object and a keyboard image.

2. Description of the Related Art

Ultrasound diagnosis apparatuses transmit ultrasound signals generated by transducers of a probe to an object and receive echo signals reflected from the object, thereby obtaining at least one image of an internal part of the object (e.g., soft tissues or blood flow). In particular, ultrasound diagnosis apparatuses are used for medical purposes including observation of the interior of an object, detection of foreign substances, and diagnosis of damage to the object. Such ultrasound diagnosis apparatuses provide high stability, display images in real time, and are safe due to the lack of radioactive exposure, compared to X-ray apparatuses. Therefore, ultrasound diagnosis apparatuses are widely used together with other image diagnosis apparatuses including a computed tomography (CT) apparatus, a magnetic resonance imaging (MRI) apparatus, and the like.

Generally, an apparatus that outputs a medical image (for example, an ultrasound image) is separated from an apparatus (for example, a keyboard) that is used for a user to input data. Therefore, it is difficult for a user to accurately input data by using a keyboard while looking at a medical image.

### SUMMARY

One or more exemplary embodiments include a method, apparatus, and system for outputting a medical image representing an object and a keyboard image.

One or more exemplary embodiments include a non-transitory computer-readable storage medium storing a program for executing the method.

Additional aspects will be set forth in part in the description which follows and, in part, will be apparent from the description, or may be learned by practice of the presented exemplary embodiments.

According to one or more exemplary embodiments, a method of outputting a medical image representing an object and a keyboard image includes: displaying the medical image and the keyboard image in different regions of a single screen; performing image processing on the medical image, based on a first user input which is input via the keyboard image; and displaying a result of the image processing on the single screen.

The result of the image processing may include an image in which a text is added to at least one portion of the medical image.

The result of the image processing may include an image which is obtained by enlarging a certain region of the medical image or an image which is obtained by reducing a certain region of the medical image.

The result of the image processing may include an image which is obtained by duplicating the medical image.

The result of the image processing may include an image which is obtained by changing a brightness of the medical image.

The displaying of the result may include simultaneously displaying the medical image and the result of the image processing on the single screen.

The keyboard image may include an image which is generated based on at least one keyboard type which is previously set.

The displaying of the medical image and the keyboard image may include displaying the keyboard image, based on a user input which is input while the medical image is displayed on the single screen.

The displaying of the medical image and the keyboard image may include displaying the medical image, based on a user input which is input while the keyboard image is displayed on the single screen.

The method may further include performing image processing on the medical image, based on a second user input which is input via the medical image, wherein the displaying of the result may include displaying a result of the image processing, performed based on the second user input, on the single screen.

The method may further include displaying at least one word, including at least one letter which is selected according to the first user input, on the single screen.

The medical image may include an image which is generated from a plurality of echo signals respectively corresponding to a plurality of ultrasound signals transmitted to the object.

According to one or more exemplary embodiments, provided is a non-transitory computer-readable storage medium storing a program for executing the method.

According to one or more exemplary embodiments, an apparatus for outputting a medical image representing an object and a keyboard image includes: an input unit that displays the medical image and the keyboard image in different regions of a single screen; and an image processor that performs image processing on the medical image, based on a first user input which is input via the keyboard image, wherein the input unit displays a result of the image processing on the single screen.

The result of the image processing may include an image in which a text is added to at least one portion of the medical image.

The result of the image processing may include an image which is obtained by enlarging a certain region of the medical image or an image which is obtained by reducing a certain region of the medical image.

The result of the image processing may include an image which is obtained by duplicating the medical image.

The result of the image processing may include an image which is obtained by changing a brightness of the medical image.

The input unit may simultaneously display the medical image and the result of the image processing on the single screen.

The keyboard image may include an image which is generated based on at least one keyboard type which is previously set.

The input unit may display the keyboard image, based on a user input which is input while the medical image is displayed on the single screen.

The input unit may display the medical image, based on a user input which is input while the keyboard image is displayed on the single screen.

The image processor may perform image processing on the medical image, based on a second user input which is input via the medical image, and the input unit may display a result of the image processing, performed based on the second user input, on the single screen.

The input unit may display at least one word, including at least one letter which is selected according to the first user input, on the single screen.

The medical image may include an image which is generated from a plurality of echo signals respectively corresponding to a plurality of ultrasound signals transmitted to the object.

According to one or more exemplary embodiments, an ultrasound diagnosis system for outputting a medical image representing an object and a keyboard image includes: a probe that transmits a plurality of ultrasound signals to the object and receives a plurality of echo signals respectively corresponding to the plurality of ultrasound signals; and an ultrasound imaging apparatus that generates the medical image by using the plurality of echo signals, displays the medical image and the keyboard image in different regions of a single screen, performs image processing on the medical image, based on a first user input which is input via the keyboard image, and displays a result of the image processing on the single screen.

### BRIEF DESCRIPTION OF THE DRAWINGS

These and/or other aspects will become apparent and more readily appreciated from the following description of the exemplary embodiments, taken in conjunction with the accompanying drawings in which:

FIGS. 1A and 1B are diagrams illustrating examples of an ultrasound diagnosis system according to an exemplary embodiment;

FIG. 2 is a block diagram illustrating an example of an ultrasound diagnosis apparatus according to an exemplary embodiment;

FIG. 3 is a block diagram illustrating an example of a wireless probe according to an exemplary embodiment;

FIG. 4 is a block diagram illustrating an example of an apparatus for outputting a medical image and an image of a keyboard, according to an exemplary embodiment;

FIG. 5 is a diagram for describing an example in which an input unit according to an exemplary embodiment outputs a medical image and an image of a keyboard;

FIG. 6 is a diagram for describing an example in which an input unit according to an exemplary embodiment outputs a recommendation word list;

FIG. 7 is a diagram for describing an example in which an input unit according to an exemplary embodiment outputs an image to which a text is added;

FIG. 8 is a diagram for describing another example in which an input unit according to an exemplary embodiment outputs an image to which a text is added;

FIG. 9 is a diagram for describing an example in which an input unit according to an exemplary embodiment outputs a spelling, selected by a user, to a separate window;

FIGS. 10A to 11B are diagrams for describing an example in which an input unit according to an exemplary embodiment outputs an image-processed result;

FIGS. 12A to 13B are diagrams for describing another example in which an input unit according to an exemplary embodiment outputs an image-processed result;

FIGS. 14A to 17B are diagrams for describing another example in which an input unit according to an exemplary embodiment outputs an image-processed result;

FIGS. 18A to 19B are diagrams for describing another example in which an input unit according to an exemplary embodiment outputs an image-processed result;

FIGS. 20A to 20D are diagrams for describing an example in which an input unit according to an exemplary embodiment outputs images of various types of keyboards;

FIGS. 21A to 23B are diagrams for describing a sequence in which a medical image and a keyboard image are output to an input unit according to an exemplary embodiment; and

FIG. 24 is a flowchart for describing an example of a method of outputting a medical image and a keyboard image, according to an exemplary embodiment.

### DETAILED DESCRIPTION

Reference will now be made in detail to exemplary embodiments, examples of which are illustrated in the accompanying drawings, wherein like reference numerals refer to like elements throughout. In this regard, the present exemplary embodiments may have different forms and should not be construed as being limited to the descriptions set forth herein. Accordingly, the exemplary embodiments are merely described below, by referring to the figures, to explain aspects of the present description.

The terms used in this specification are those general terms currently widely used in the art in consideration of functions regarding the inventive concept, but the terms may vary according to the intention of those of ordinary skill in the art, precedents, or new technology in the art. Also, some terms may be arbitrarily selected by the applicant, and in this case, the meaning of the selected terms will be described in detail in the detailed description of the present specification. Thus, the terms used in the specification should be understood not as simple names but based on the meaning of the terms and the overall description of the invention.

Throughout the specification, it will also be understood that when a component “includes” an element, unless there is another opposite description thereto, it should be understood that the component does not exclude another element and may further include another element. In addition, terms such as “ . . . unit”, “ . . . module”, or the like refer to units that perform at least one function or operation, and the units may be implemented as hardware or software or as a combination of hardware and software.

Throughout the specification, an “ultrasound image” refers to an image of an object, or an image which represents a region of interest (ROI) included in an object and is obtained using ultrasound waves. Here, the ROI is a region which a user desires to carefully observe in the object, and for example, may be a lesion. Furthermore, an “object” may be a human, an animal, or a part of a human or animal. For example, the object may be an organ (e.g., the liver, heart, womb, brain, breast, or abdomen), a blood vessel, or a combination thereof. Also, the object may be a phantom. The phantom means a material having a density, an effective atomic number, and a volume that are approximately the same as those of an organism. For example, the phantom may be a spherical phantom having properties similar to a human body.

Throughout the specification, a “user” may be, but is not limited to, a medical expert, for example, a medical doctor, a nurse, a medical laboratory technologist, or a medical imaging expert, or a technician who repairs medical apparatuses.

Exemplary embodiments now will be described more fully hereinafter with reference to the accompanying drawings, in which illustrative embodiments of the invention are shown.

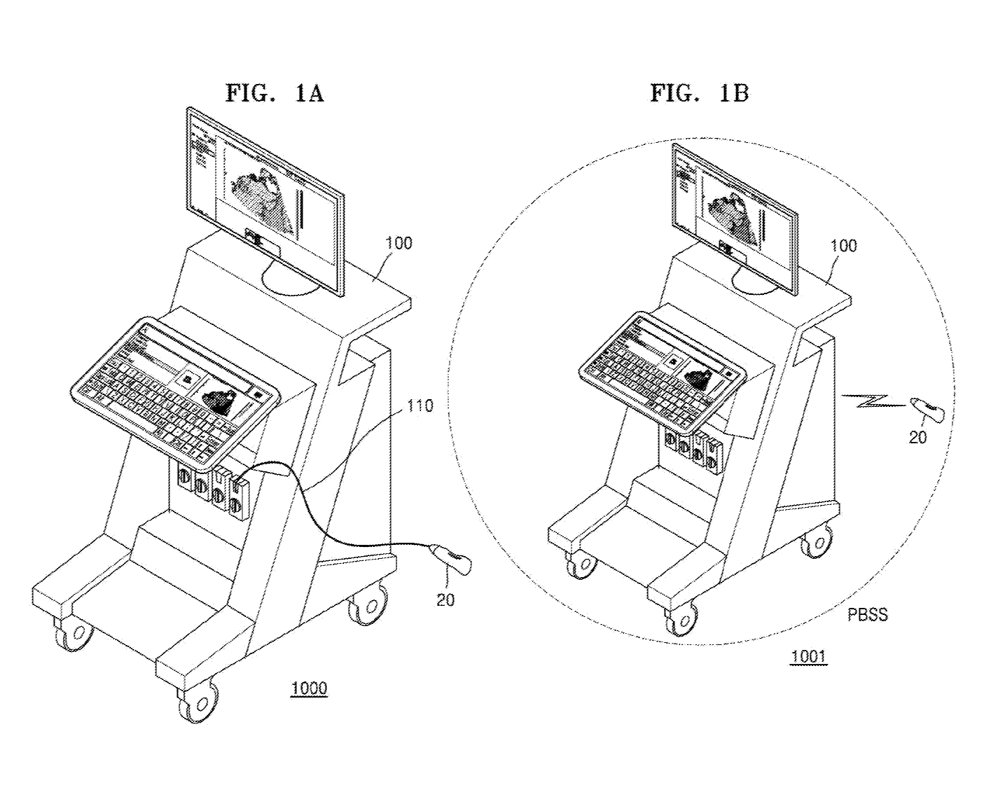

FIGS. 1A and 1B are diagrams illustrating examples of an ultrasound diagnosis system according to an exemplary embodiment.

Referring to FIG. 1A, in an ultrasound diagnosis system 1000, a probe 20 may be connected to an ultrasound imaging apparatus 100 by wire. In other words, the probe 20 which transmits or receives an ultrasound wave may be connected to a body of the ultrasound diagnosis system 1000 through a cable 110, namely, the ultrasound imaging apparatus 100.

Referring to FIG. 1B, in an ultrasound diagnosis system 1001, a probe 20 may be wirelessly connected to an ultrasound imaging apparatus 100. In other words, the probe 20 may be connected to the ultrasound imaging apparatus 100 over the same wireless network. For example, the probe 20 and the ultrasound imaging apparatus 100 may be connected to a millimeter wave (mmWave)-based wireless network, and may transmit an echo signal, received through a transducer, to the ultrasound imaging apparatus 100 at a frequency band of 60 GHz. Also, the ultrasound imaging apparatus 100 may generate ultrasound images having various modes by using the echo signal which is received at the frequency band of 60 GHz, and display the generated ultrasound image. Here, the millimeter wave-based wireless network may use a wireless communication method based on the WiGig standard of wireless gigabit alliance, but is not limited thereto.

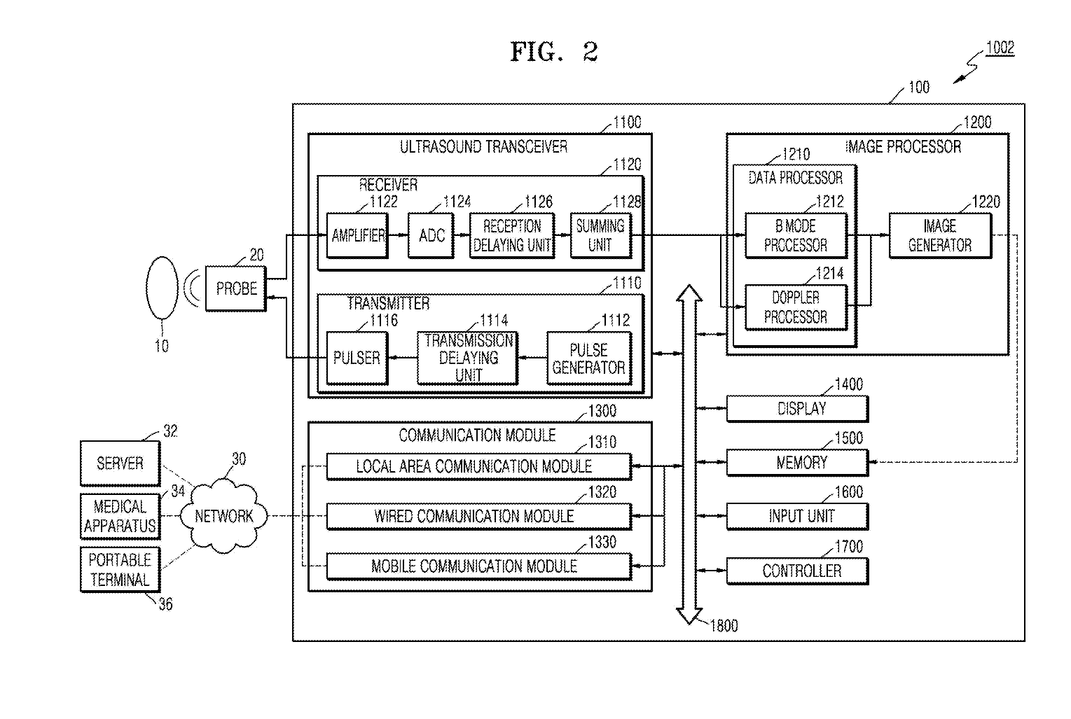

FIG. 2 is a block diagram illustrating an example of an ultrasound diagnosis apparatus according to an exemplary embodiment.

Referring to FIG. 2, an ultrasound diagnosis system 1002 may include a probe 20 and an ultrasound imaging apparatus 100. The ultrasound imaging apparatus 100 may include an ultrasound transceiver 1100, an image processor 1200, a communication module 1300, a display 1400, a memory 1500, an input unit 1600, and a controller 1700, which may be connected to one another via buses 1800.

The ultrasound diagnosis system 1002 may be a cart type apparatus or a portable type apparatus. Examples of portable ultrasound diagnosis apparatuses may include, but are not limited to, a picture archiving and communication system (PACS) viewer, a smartphone, a laptop computer, a personal digital assistant (PDA), and a tablet PC.

The probe 20 transmits an ultrasound signal to an object 10 (or an ROI of the object 10) according to a driving signal applied from the ultrasound transceiver 1100, and receives an echo signal reflected from the object 10 (or the ROI of the object 10). The probe 20 includes a plurality of transducers, and the plurality of transducers oscillate in response to electric signals and generate acoustic energy, that is, ultrasound waves. Furthermore, the probe 20 may be connected to the main body of the ultrasound diagnosis system 1002 by wire or wirelessly, and according to embodiments, the ultrasound diagnosis system 1002 may include a plurality of probes 20.

A transmitter 1110 supplies a driving signal to the probe 20. The transmitter 110 includes a pulse generator 1112, a transmission delaying unit 1114, and a pulser 1116. The pulse generator 1112 generates pulses for forming transmission ultrasound waves based on a predetermined pulse repetition frequency (PRF), and the transmission delaying unit 1114 delays the pulses by delay times necessary for determining transmission directionality. The pulses which have been delayed correspond to a plurality of piezoelectric vibrators included in the probe 20, respectively. The pulser 1116 applies a driving signal (or a driving pulse) to the probe 20 based on timing corresponding to each of the pulses which have been delayed.

A receiver 1120 generates ultrasound data by processing echo signals received from the probe 20. The receiver 120 may include an amplifier 1122, an analog-to-digital converter (ADC) 1124, a reception delaying unit 1126, and a summing unit 1128. The amplifier 1122 amplifies echo signals in each channel, and the ADC 1124 performs analog-to-digital conversion with respect to the amplified echo signals. The reception delaying unit 1126 delays digital echo signals output by the ADC 124 by delay times necessary for determining reception directionality, and the summing unit 1128 generates ultrasound data by summing the echo signals processed by the reception delaying unit 1166. In some embodiments, the receiver 1120 may not include the amplifier 1122. In other words, if the sensitivity of the probe 20 or the capability of the ADC 1124 to process bits is enhanced, the amplifier 1122 may be omitted.

The image processor 1200 generates an ultrasound image by scan-converting ultrasound data generated by the ultrasound transceiver 1100. The ultrasound image may be not only a grayscale ultrasound image obtained by scanning an object in an amplitude (A) mode, a brightness (B) mode, and a motion (M) mode, but also a Doppler image showing a movement of an object via a Doppler effect. The Doppler image may be a blood flow Doppler image showing flow of blood (also referred to as a color Doppler image), a tissue Doppler image showing a movement of tissue, or a spectral Doppler image showing a moving speed of an object as a waveform.

A B mode processor 1212 extracts B mode components from ultrasound data and processes the B mode components. An image generator 1220 may generate an ultrasound image indicating signal intensities as brightness based on the extracted B mode components 1212.

Similarly, a Doppler processor 1214 may extract Doppler components from ultrasound data, and the image generator 1220 may generate a Doppler image indicating a movement of an object as colors or waveforms based on the extracted Doppler components.

According to an embodiment, the image generator 1220 may generate a three-dimensional (3D) ultrasound image via volume-rendering with respect to volume data and may also generate an elasticity image by imaging deformation of the object 10 due to pressure. Furthermore, the image generator 1220 may display various pieces of additional information in an ultrasound image by using text and graphics. In addition, the generated ultrasound image may be stored in the memory 1500.

The display 1400 displays the generated ultrasound image. The display 1400 may display not only an ultrasound image, but also various pieces of information processed by the ultrasound diagnosis apparatus 1002 on a screen image via a graphical user interface (GUI). In addition, the ultrasound diagnosis apparatus 1000 may include two or more the displays 1400 according to embodiments.

The communication module 1300 is connected to a network 30 by wire or wirelessly to communicate with an external device or a server. Also, when the probe 20 is connected to the ultrasound imaging apparatus 1002 over a wireless network, the communication module 1300 may communicate with the probe 20.

The communication module 1300 may exchange data with a hospital server or another medical apparatus in a hospital, which is connected thereto via a PACS. Furthermore, the communication module 1300 may perform data communication according to the digital imaging and communications in medicine (DICOM) standard.

The communication module 1300 may transmit or receive data related to diagnosis of an object, e.g., an ultrasound image, ultrasound data, and Doppler data of the object, via the network 30 and may also transmit or receive medical images captured by another medical apparatus, e.g., a computed tomography (CT) apparatus, a magnetic resonance imaging (MRI) apparatus, or an X-ray apparatus. Furthermore, the communication module 1300 may receive information about a diagnosis history or medical treatment schedule of a patient from a server and utilizes the received information to diagnose the patient. Furthermore, the communication module 1300 may perform data communication not only with a server or a medical apparatus in a hospital, but also with a portable terminal of a medical doctor or patient.

The communication module 1300 is connected to the network 30 by wire or wirelessly to exchange data with a server 32, a medical apparatus 34, or a portable terminal 36. The communication module 1300 may include one or more components for communication with external devices. For example, the communication module 1300 may include a local area communication module 1310, a wired communication module 1320, and a mobile communication module 1330.

The local area communication module 1310 refers to a module for local area communication within a predetermined distance. Examples of local area communication techniques according to an embodiment may include, but are not limited to, wireless LAN, Wi-Fi, Bluetooth, ZigBee, Wi-Fi Direct (WFD), ultra wideband (UWB), infrared data association (IrDA), Bluetooth low energy (BLE), and near field communication (NFC).

The wired communication module 1320 refers to a module for communication using electric signals or optical signals. Examples of wired communication techniques according to an embodiment may include communication via a twisted pair cable, a coaxial cable, an optical fiber cable, and an Ethernet cable.

The mobile communication module 1330 transmits or receives wireless signals to or from at least one selected from a base station, an external terminal, and a server on a mobile communication network. The wireless signals may be voice call signals, video call signals, or various types of data for transmission and reception of text/multimedia messages.

The memory 1500 stores various data processed by the ultrasound diagnosis apparatus 1000. For example, the memory 1500 may store medical data related to diagnosis of an object, such as ultrasound data and an ultrasound image that are input or output, and may also store algorithms or programs which are to be executed in the ultrasound imaging apparatus 1002.

The memory 1500 may be any of various storage media, e.g., a flash memory, a hard disk drive, EEPROM, etc. Furthermore, the ultrasound imaging apparatus 1002 may utilize web storage or a cloud server that performs the storage function of the memory 1500 online.

The input unit 1600 refers to a means via which a user inputs data for controlling the ultrasound imaging apparatus 1002. Example of the input unit 1600 may include hardware elements, such as a keyboard, a mouse, a touch pad, a touch screen, a trackball, and a jog switch, and a software module for operating the hardware elements. However, embodiments are not limited thereto, and the input unit 1600 may further include any of various other input units including an electrocardiogram (ECG) measuring module, a respiration measuring module, a voice recognition sensor, a gesture recognition sensor, a fingerprint recognition sensor, an iris recognition sensor, a depth sensor, a distance sensor, etc.

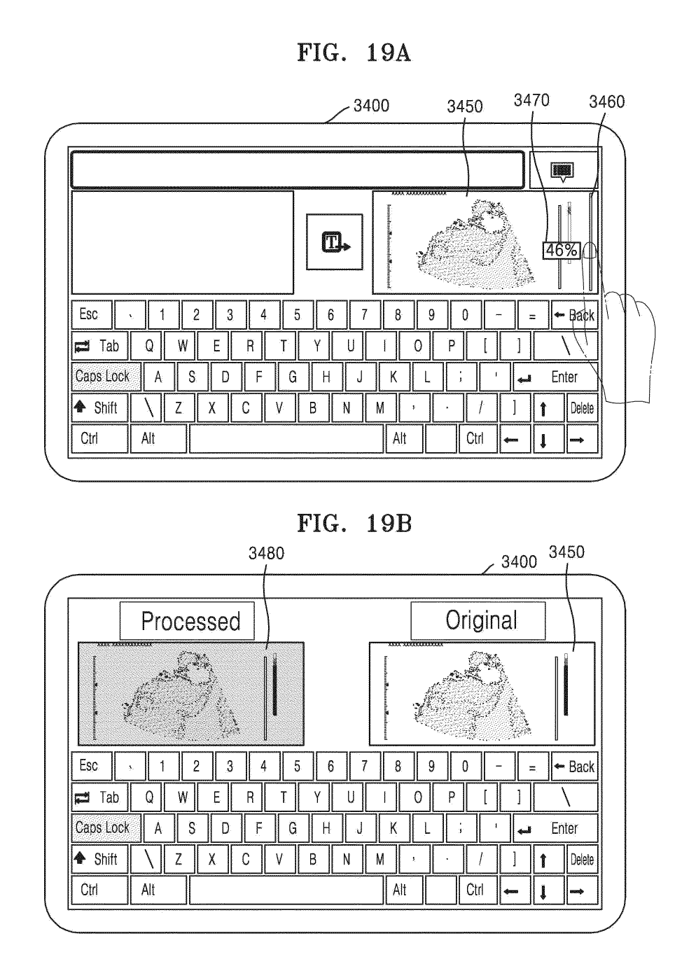

The input unit 1600 according to an exemplary embodiment may output an ultrasound image, representing the object 10 (or the ROI of the object 10), and a keyboard image. That is, the input unit 1600 may include a single touch screen and a software module for operating the single touch screen, and the input unit 1600 may output the ultrasound image and the keyboard image to the single touch screen. Here, the keyboard image denotes an image where a keyboard, which receives, from a user, data (i.e., a user input) for controlling the ultrasound imaging apparatus 1002, is displayed on a touch screen. For example, the keyboard image may be an image in which keys included in a general keyboard are displayed. As another example, the keyboard image may be an image which is generated based on a predetermined keyboard type. Detailed examples of the keyboard image will be described below with reference to FIGS. 19A to 19D.

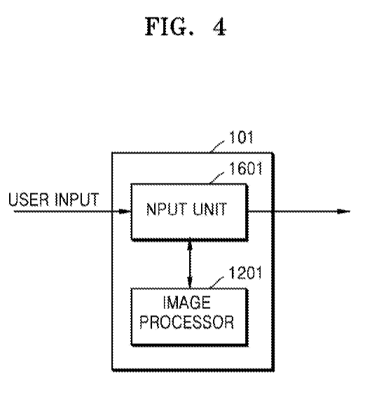

The image processor 1200 performs image processing on an ultrasound image, based on a user input to the keyboard image. The input unit 1600 outputs a result of the image processing (i.e., a processed image) to the single touch screen. Therefore, in a case where the user selects a desired key from the keyboard, an inconvenience of alternately looking at the medical image and the keyboard displayed by the display 1400 is avoided. The input unit 1600 and the image processor 1200 according to an exemplary embodiment will be described below in detail with reference to FIG. 4.

The controller 1700 may control all operations of the ultrasound diagnosis apparatus 1000. In other words, the controller 1700 may control operations among the probe 20, the ultrasound transceiver 1100, the image processor 1200, the communication module 1300, the display 1400, the memory 1500, and the input unit 1600 shown in FIG. 1.

All or some of the probe 20, the ultrasound transceiver 1100, the image processor 1200, the communication module 1300, the display 1400, the memory 1500, the input unit 1600, and the controller 1700 may be implemented as software modules. Furthermore, at least one selected from the ultrasound transceiver 1100, the image processor 1200, and the communication module 1300 may be included in the controller 1600. However, embodiments of the present invention are not limited thereto.

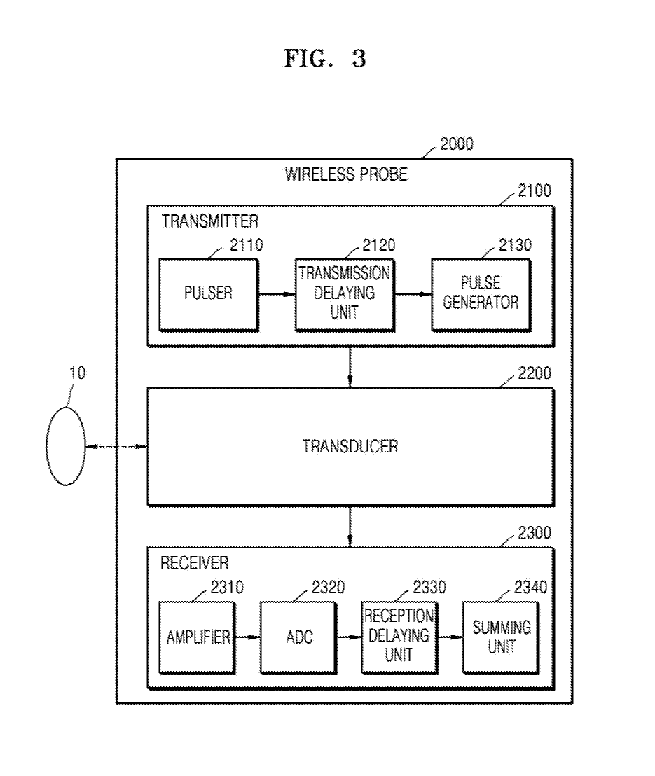

FIG. 3 is a block diagram illustrating an example of a wireless probe according to an exemplary embodiment.

Referring to FIG. 3, a wireless probe 2000 includes a plurality of transducers as described above with reference to FIG. 2, and may include all or some of the elements of the ultrasound transceiver 1100 depending on an implementation type.

The wireless probe 2000 according to the embodiment shown in FIG. 3 includes a transmitter 2100, a transducer 2200, and a receiver 2300. Since descriptions thereof are given above with reference to FIG. 2, detailed descriptions thereof will be omitted here. In addition, according to embodiments, the wireless probe 2000 may selectively include a reception delaying unit 2330 and a summing unit 2340.

The wireless probe 2000 may transmit ultrasound signals to the object 10, receive echo signals from the object 10, generate ultrasound data, and wirelessly transmit the ultrasound data to the ultrasound imaging apparatus 1002 shown in FIG. 2.

FIG. 4 is a block diagram illustrating an example of an apparatus for outputting a medical image and an image of a keyboard, according to an exemplary embodiment.

Referring to FIG. 4, an apparatus 101 includes an input unit 1601 and an image processor 1201. Here, all or one of the input unit 1601 and the image processor 1201 may be operated by a software module, but the present embodiment is not limited thereto. Some of the above-described elements may be operated by hardware. Also, each of the input unit 1601 and the image processor 1201 may include a control module, and the apparatus 101 may include a separate control module that controls the input unit 1601 and the image processor 1201.

Moreover, the input unit 1601 may be the same as the input unit 1600 of FIG. 2, and the image processor 1201 may be the same as the image processor 1200 of FIG. 2. If the apparatus 101 is an ultrasound imaging apparatus, the apparatus 101 may further include the ultrasound transceiver 1100, the communication module 1300, the display 1400, the memory 1500, and the controller 1700 illustrated in FIG. 2, in addition to the input unit 1601 and the image processor 1201.

The input unit 1601 displays a medical image and a keyboard image in different regions of a single screen. For example, the medical image may be an ultrasound image which represents an object 10 (or an ROI of the object 10), but is not limited thereto. The medical image may include various kinds of images such as a magnetic resonance (MR) image, an X-ray image, a CT image, a position emission tomography (PET) image, and an optical coherence tomography (OCT) image, in addition to the ultrasound image.

The keyboard image may be an image which represent keys included in a general keyboard, but is not limited thereto. For example, the keyboard image may be an image which is generated based on a predetermined keyboard type. In this case, the keyboard type may be set by a user or may be set by a manufacturer or a seller of the apparatus 101. For example, the keyboard type may be a shape of a keyboard or a type in which a color is changed. As another example, the keyboard type may be a type in which keys included in a keyboard are changed. As another example, the keyboard type may be a type in which shortcut keys respectively corresponding to functions are combined.

The input unit 1601 receives a user input. Here, the user input may be input to the keyboard image. For example, when the user touches one or more keys in the keyboard image displayed by the input unit 1601, the input unit 1601 may receive a user input.

Moreover, the user input may be input to the medical image. For example, when a user applies a gesture to the medical image displayed by the input unit 1601, the input unit 1601 may receive the user input. Examples of the gesture described herein may include a tap, a touch and hold, a double tap, a drag, panning, a flick, a drag and drop, a pinch, and a stretch.

The image processor 1201 performs image processing on the medical image, based on the user input. For example, the image processor 1201 may add a text to a portion of the medical image. As another example, the image processor 1201 may enlarge or reduce a certain region of the medical image. As another example, the image processor 1201 may change (or adjust) a brightness of the medical image. As another example, the image processor 1201 may duplicate a pre-generated medical image.

The input unit 1601 outputs a result (i.e., an image for which image processing has been performed) of the image processing performed by the image processor 1201. At this time, the input unit 1601 may display both a before-image-processing image and an image-processing-performed image. Also, the input unit 1601 may display a plurality of images and an image which is selected from among the plurality of images by the user. Also, the input unit 1601 may display words, including a spelling which is selected according to a user input, along with the medical image and the keyboard image.

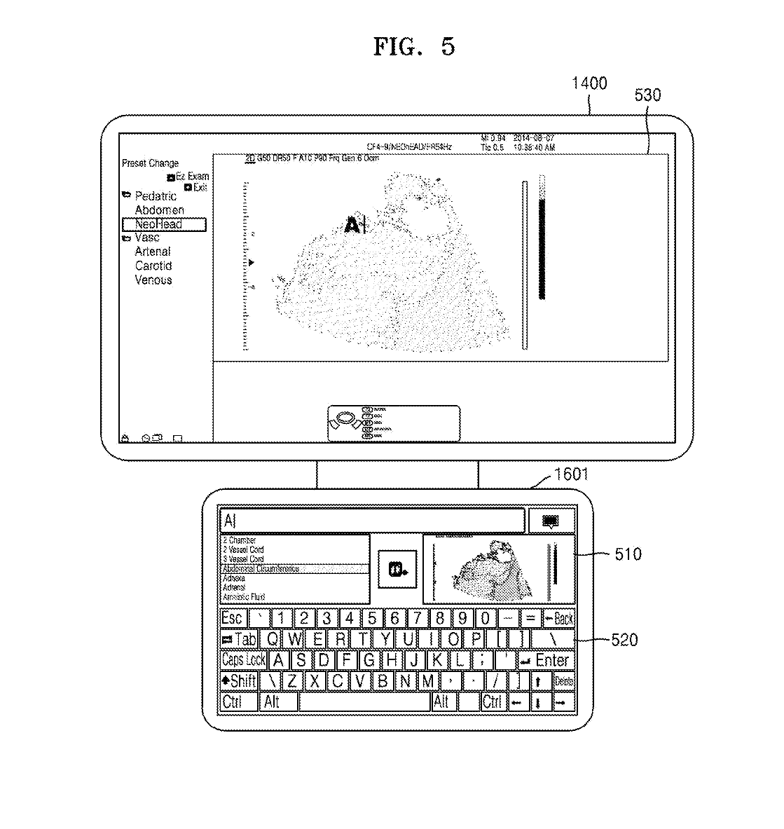

Hereinafter, examples in which the input unit 1601 outputs a medical image and a keyboard image and outputs a result of image processing performed by the image processor 1201 will be described in detail with reference to FIGS. 5 to 18B.

FIG. 5 is a diagram for describing an example in which an input unit according to an exemplary embodiment outputs a medical image and an image of a keyboard.

In FIG. 5, an example of the input unit 1601 is illustrated. Also, for convenience of a description, the display 1400 connected to the input unit 1601 is illustrated along with the input unit 1601.

Generally, the display 1400 is provided separately from the input unit 1601, and for this reason, there is inconvenience in which a user inputs data through the input unit 1601 while looking at a medical image 530 displayed by the display 1400. For example, in a case of inputting data with eyes being fixed to the medical image 530, an error occurs by selecting an undesired key. As another example, in a case of inputting data with eyes being fixed to the medical image 530, image processing may be performed for an undesired image.

The input unit 1601 according to an exemplary embodiment displays a medical image 510 and a keyboard image 520 on a single screen. Therefore, eyes of a user are not dispersed, and thus, the user may input data so that image processing is accurately performed for a desired image. Here, the medical image 510 displayed by the input unit 1601 may be the same as or differ from the medical image 530 displayed by the display 1400.

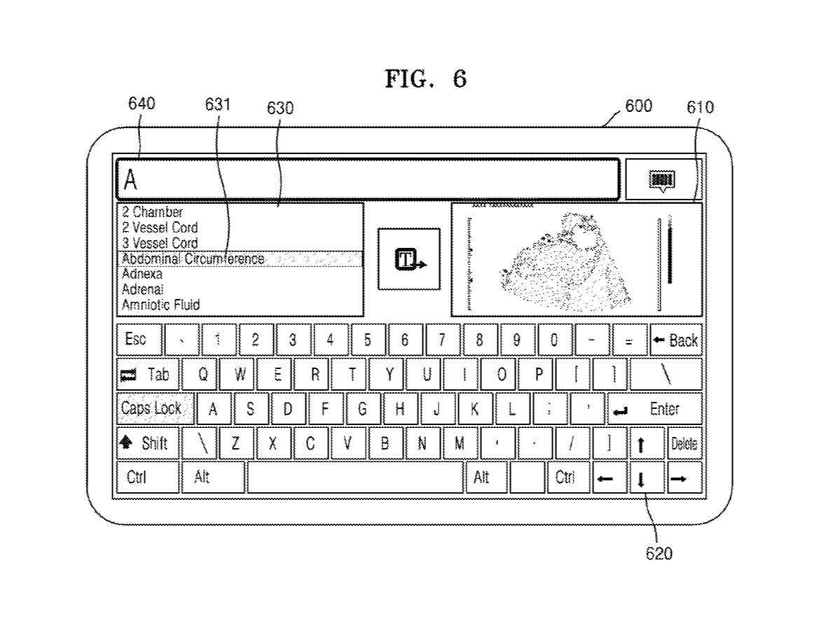

The input unit 1601 may display a word, including a spelling input by the user, on the single screen which the medical image 510 and the keyboard image 520 are displayed on. Hereinafter, this will be described in detail with reference to FIG. 6.

FIG. 6 is a diagram for describing an example in which an input unit according to an exemplary embodiment outputs a recommendation word list.

In FIG. 6, an example of a screen 600 which a medical image 610 and a keyboard image 620 are displayed on is illustrated. Also, a recommendation word list 630 is displayed on the screen 600. Here, the recommendation word list 630 denotes a list including words which are to be input by a user.

The input unit 1601 may select one word from among the words included in the recommendation word list 630, based on a user input. For example, the input unit 1601 may select a word by using a spelling corresponding to a key which is selected by the user from among the words included in the keyboard image 620. If the user touches a key corresponding to “A”, the input unit 1601 may select a word, including “A”, from among the words included in the recommendation word list 630. For example, the input unit 1610 may select a word, which has “A” as a first spelling, from among the words included in the recommendation word list 630.

At this time, if the user continuously touches two or more keys, the input unit 1601 may select a word, including spellings which are sequentially input, from the recommendation word list 630. For example, if the user sequentially touches a key corresponding to “A” and a key corresponding to “B”, the input unit 1601 may select a word including “AB”. For example, the input unit 1601 may select a word, which has “AB” as first two spellings, from among the words included in the recommendation word list 630.

Moreover, the input unit 1601 may display (631) the selected word in a shape or a color which differ from those of other words. Therefore, the user easily identifies a word which is selected from among a plurality of words by the input unit 1601. Also, the input unit 1601 may display a spelling, which is selected (for example, a key is touched) by the user from among keys included in the keyboard image 620, in one region of the screen 600.

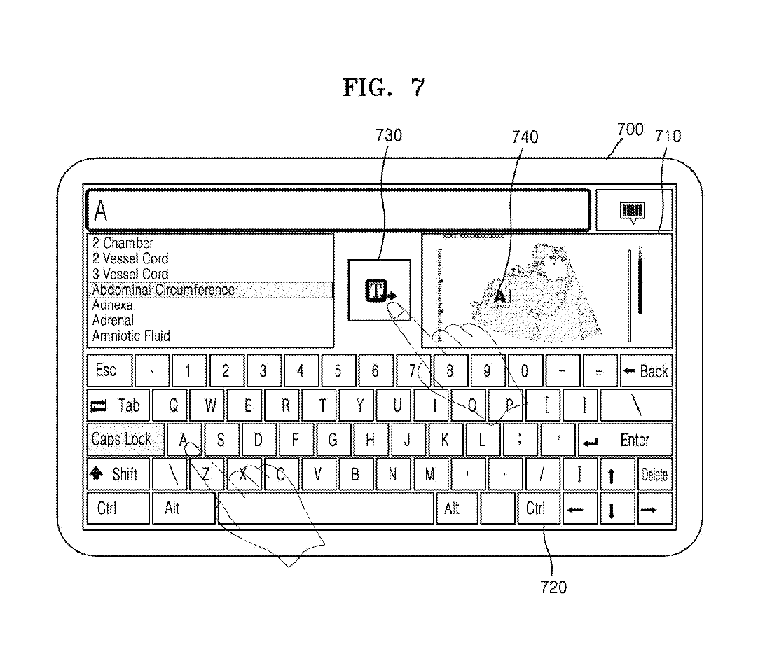

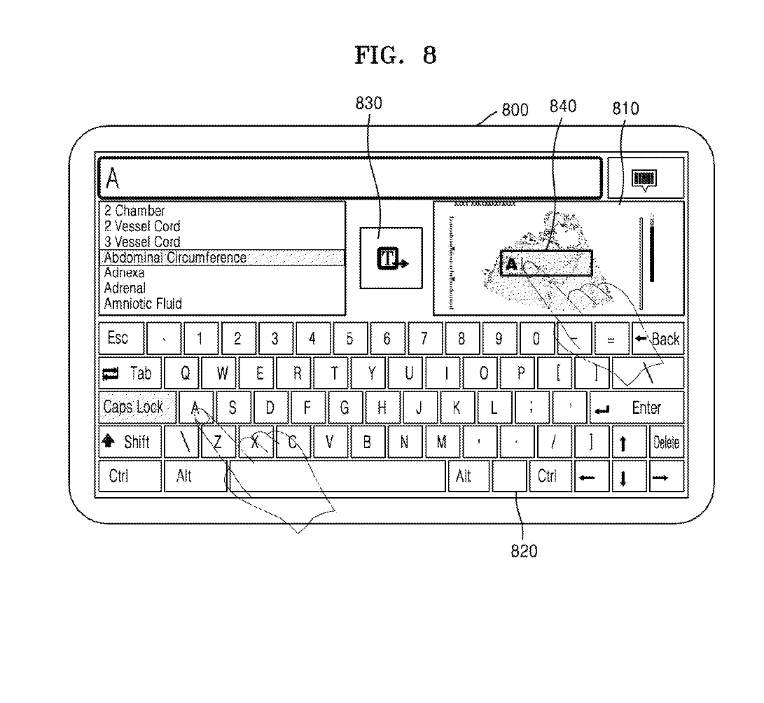

The image processor 1201 may add a text to at least one portion of the medical image, based on a user input, and the input unit 1601 may display an image to which the text is added. Here, the text may include a number and a sign in addition to a spelling which constitutes a letter. Hereinafter, examples in which the image processor 1201 adds a text to a medical image and the input unit 1601 displays an image with a text added thereto will be described in detail with reference to FIGS. 7 and 8.

FIG. 7 is a diagram for describing an example in which an input unit according to an exemplary embodiment outputs an image to which a text is added.

In FIG. 7, an example of a screen 700 which an image 710 with a text 740 added thereto and a keyboard image 720 are displayed on is illustrated.

The input unit 1601 may receive a user input which adds the text 740 to a medical image. For example, when a user selects (for example, touch) an icon 730 displayed in one region of the screen 700, the text 740 input by the user may added to the medical image.

When the user inputs the text 740 through the keyboard image 720 after selecting the icon 730, the image processor 1201 adds the input text 740 to the medical image. For example, if the user touches a key corresponding to “A” among keys included in the keyboard image 720 after selecting the icon 730, the image processor 1201 adds “A” to the medical image. For example, the image processor 1201 may generate a new image, to which the text 740 is added, in one region of the medical image.

In this case, a region to which the text 740 is added may be designated by the user, and the image processor 1201 may be automatically selected. For example, when the user touches a point, to which the text 740 is to be added, in the medical image currently displayed on the screen 700, the image processor 1201 may add the text 740 to the point touched by the user. As another example, without intervention of the user, the image processor 1201 may add the text 740 to a central region of the medical image currently displayed on the screen 700.

The image processor 1201 transmits a result (i.e., the image 710 to which the text 740 is added) of image processing to the input unit 1601. The input unit 1601 displays the image 710, to which the text 710 is added, in one region of the screen 700.

In FIG. 7, it is illustrated that one spelling 740 is added to the medical image, but the present embodiment is not limited thereto. In other words, a word, a phrase, or a sentence in which a plurality of spellings are combined may be added to the medical image.

Even though the user does not select the icon 730 displayed on the screen 700, the input unit 1601 may add the text 740 to the medical image. Hereinafter, another example in which the input unit 1601 adds the text 740 to the medical image will be described with reference to FIG. 8.

FIG. 8 is a diagram for describing another example in which an input unit according to an exemplary embodiment outputs an image to which a text is added.

In FIG. 8, an example of an image 810 to which a text is added and a screen 800 which a keyboard image 820 is displayed on is illustrated.

A user may add a text to a medical image even without selecting an icon 830 displayed on the screen. When the user continuously inputs letters after selecting a position 840 to which a text is to be added in the medical image, the input unit 1601 may issue a request, to the image processor 1201, to add an input letter to the position touched by the user. For example, if the user touches the position 840 to which a text is to be input in the medical image and touches a key corresponding to “A” among keys included in the keyboard image 820, the image processor 1201 may add “A” to the designated position of the medical image.

The image processor 1201 transmits a result (i.e., the image 810 to which the text is added) of image processing to the input unit 1601. The input unit 1601 displays the image 810, to which the text is added, in one region of the screen 800.

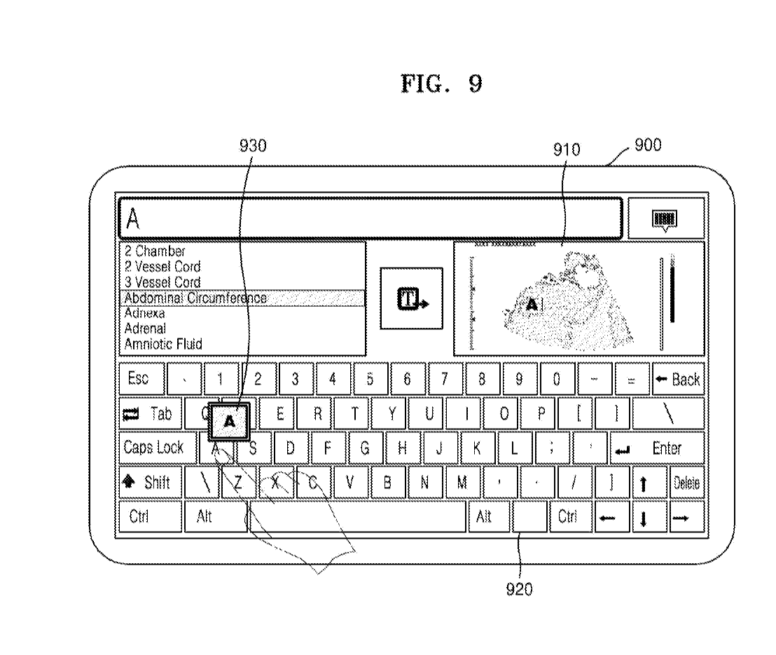

FIG. 9 is a diagram for describing an example in which an input unit according to an exemplary embodiment outputs a spelling, selected by a user, to a separate window.

In FIG. 9, an example of an image 910 to which a text is added and a screen 900 which a keyboard image 920 is displayed on is illustrated.

In the keyboard image 920, a boundary between adjacent keys may be unclear unlike a physical keyboard. Therefore, when a user touches a key, a key adjacent to a desired key may be touched.

When the user selects one key included in the keyboard image 920, the input unit 1601 may display a spelling, corresponding to the selected key, in a separate window 930. For example, if the user touches a key corresponding to “A” among keys included in the keyboard image 920, the input unit 1601 may display the window 930, in which “A” is displayed, on the screen 900 for a certain time immediately after the user touches the key.

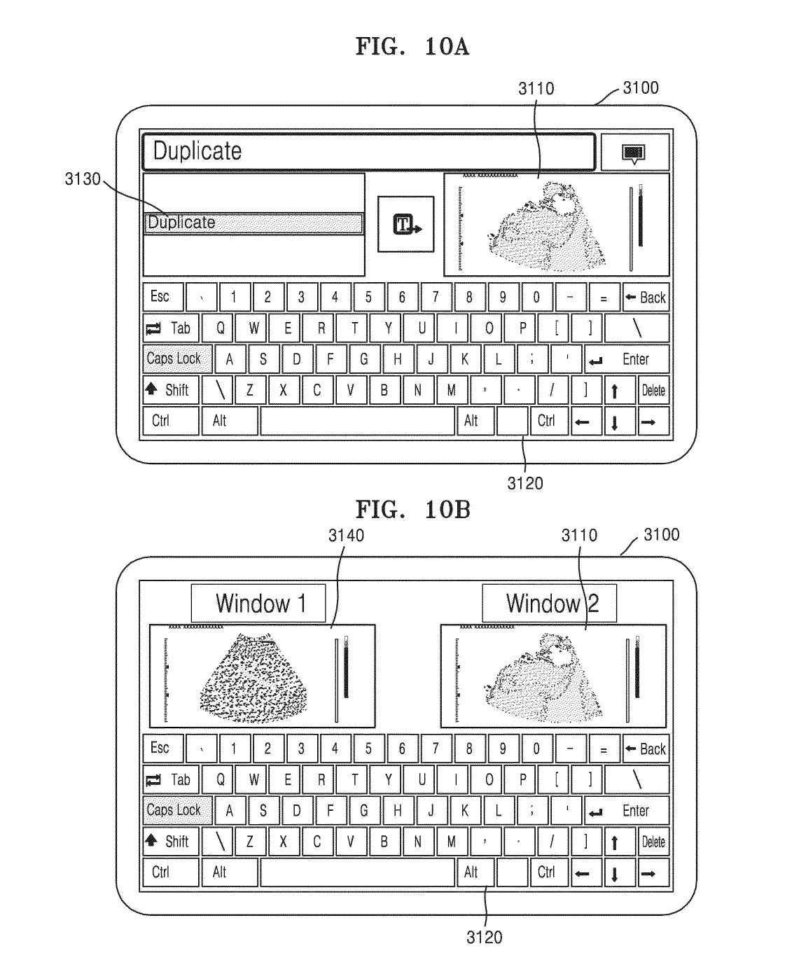

As described above with reference to FIGS. 8 and 9, the input unit 1601 may display an image, to which a text input by the user is added, on a screen. In addition to a text being added to a medical image, the image processor 1201 may perform image processing on a medical image displayed on a screen in various schemes, based on a user input. Hereinafter, examples in which the image processor 1201 performs image processing and the input unit 1601 displays an image-processed result will be described with reference to FIGS. 10A to 19B.

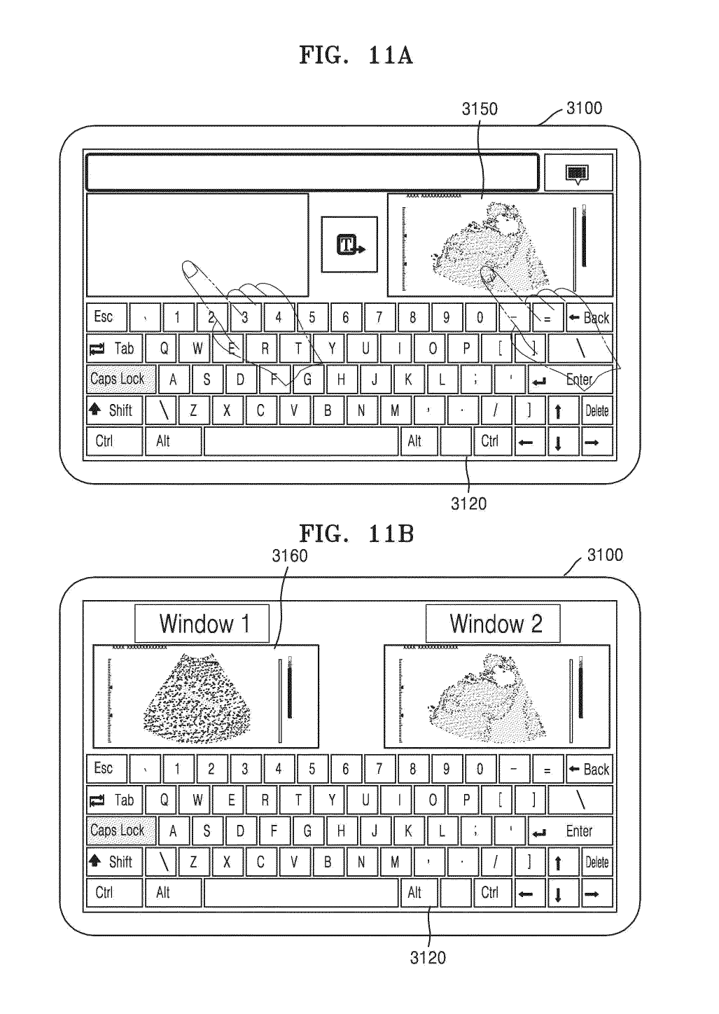

FIGS. 10A to 11B are diagrams for describing an example in which an input unit according to an exemplary embodiment outputs an image-processed result.

The input unit 1601 may receive a user input which requests duplication of a medical image 3110 displayed on a screen 3100, and the image processor 1201 may duplicate the medical image 3110, based on a user input. Here, duplicating the medical image 3110 may denote that the image processor 1201 further generates the same image as the medical image 3110, or denote that the input unit 1601 displays one more image, which is the same as the medical image 3110, on the screen 3100.

Referring to FIG. 10A, a user may input data through a keyboard image 3120. For example, if the term which means duplication of the medical image 3110 is “duplicate”, the medical image 3110 may be duplicated by the user inputting “d”, “u”, “p”, “I”, “i”, “c”, “a”, “t”, and “e” through the keyboard image 3120. Also, the user may select “duplicate” from a recommendation word list 3130 displayed on the screen 3100.

Referring to FIG. 10B, an image 3140 which is the same as the medical image 3110 displayed on the screen 3100 may be displayed along with the medical image 3110. Here, the newly displayed image 3140 and the displayed image 3110 may be displayed in different regions of the screen 3100.

Referring to FIG. 11A, the user may duplicate a medical image 3150 by applying a gesture to the screen 3100. For example, the medical image 3150 may be duplicated by the user dragging and dropping the medical image 3150. The gesture is not limited to a drag and drop, and the medical image 3150 may be duplicated by the user making another predetermined gesture.

Referring to FIG. 11B, an image 3160 which is the same as the medical image 3110 displayed on the screen 3100 may be displayed along with the medical image 3110. Here, the newly displayed image 3160 and the displayed image 3110 may be displayed in different regions of the screen 3100.

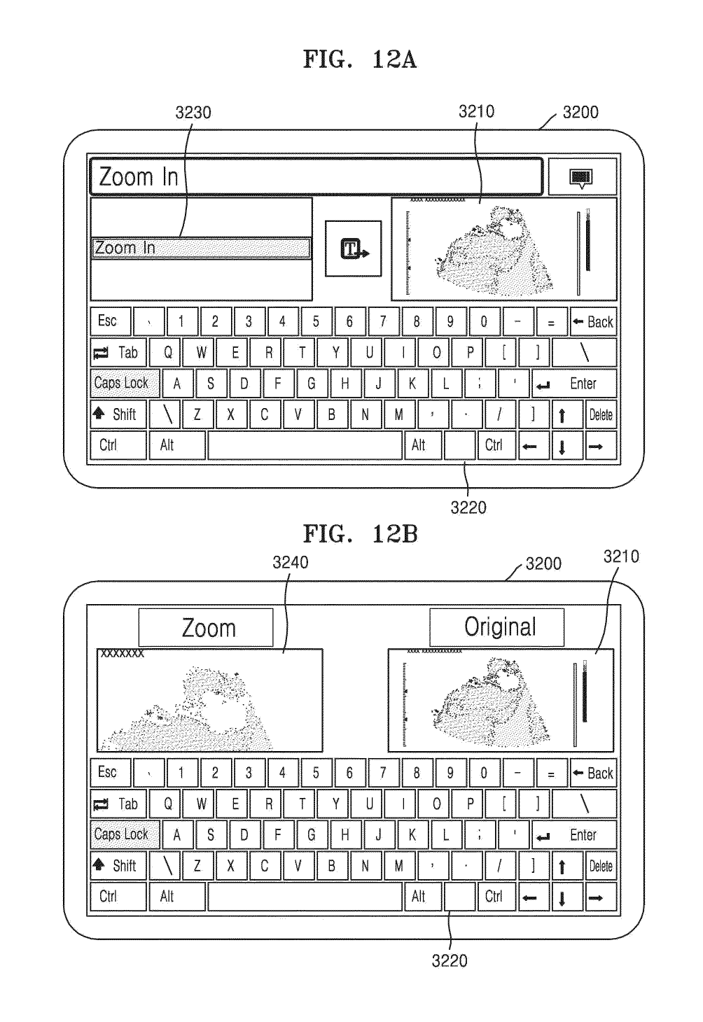

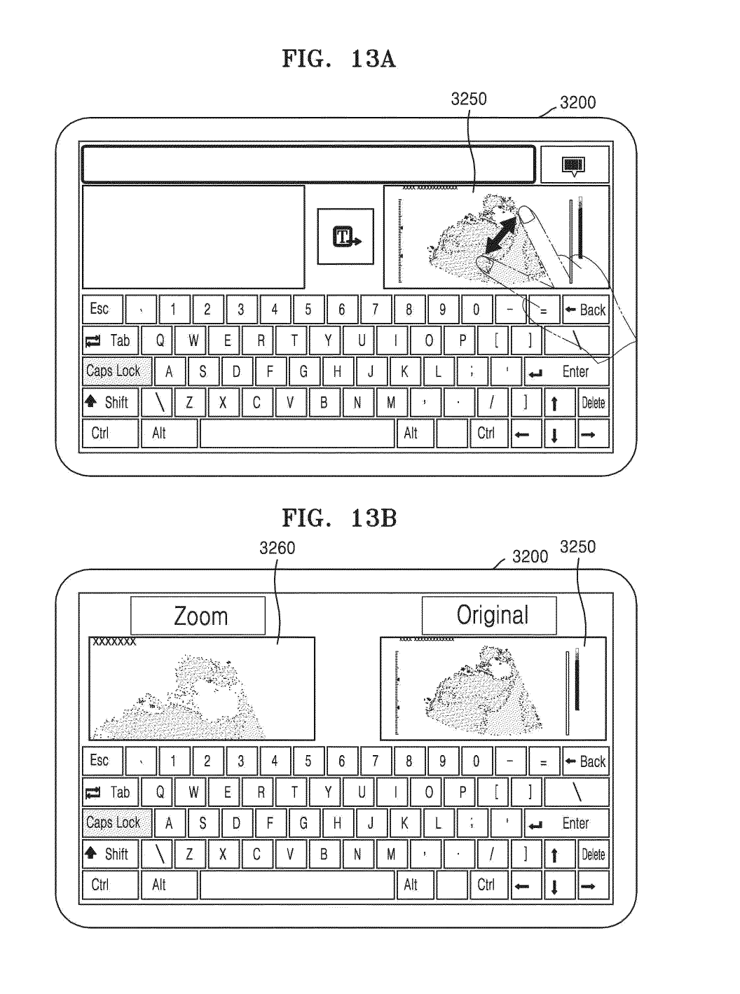

FIGS. 12A to 13B are diagrams for describing another example in which an input unit according to an exemplary embodiment outputs an image-processed result.

The input unit 1601 may receive a user input that requests enlargement or reduction of a medical image 3210 displayed on a screen 3200, and the image processor 1201 may generate an image which is obtained by enlarging or reducing the medical image 3210, based on a user input. Hereinafter, only an example in which the image processor 1201 generates an image which is obtained by enlarging the medical image 3210 will be described, but an example in which the image processor 1201 generates an image which is obtained by reducing the medical image 3210 may be understood by one of ordinary skill in the art.

Referring to FIG. 12A, a user may input data through a keyboard image 3220. For example, if the term which means duplication of the medical image 3210 is “zoom in”, the medical image 3210 may be enlarged by the user inputting “z”, “0”, “0”, “m”, “i”, and “n” through the keyboard image 3220. Also, the user may select “zoom in” from a recommendation word list 3230 displayed on the screen 3200.

Referring to FIG. 12B, an image 3240 which is the same as the medical image 3210 displayed on the screen 3200 may be displayed along with the medical image 3210. Here, the newly displayed image 3240 and the displayed image 3210 may be displayed in different regions of the screen 3200.

Referring to FIG. 13A, the user may duplicate a medical image 3250 by applying a gesture to the screen 3200. For example, the medical image 3150 may be duplicated by the user applying a stretch gesture to the medical image 3150. The gesture is not limited to a stretch, and the medical image 3150 may be duplicated by the user making another predetermined gesture.

Referring to FIG. 13B, an image 3260 which is the same as the medical image 3250 displayed on the screen 3100 may be displayed along with the medical image 3250. Here, the newly displayed image 3260 and the displayed image 3250 may be displayed in different regions of the screen 3200.

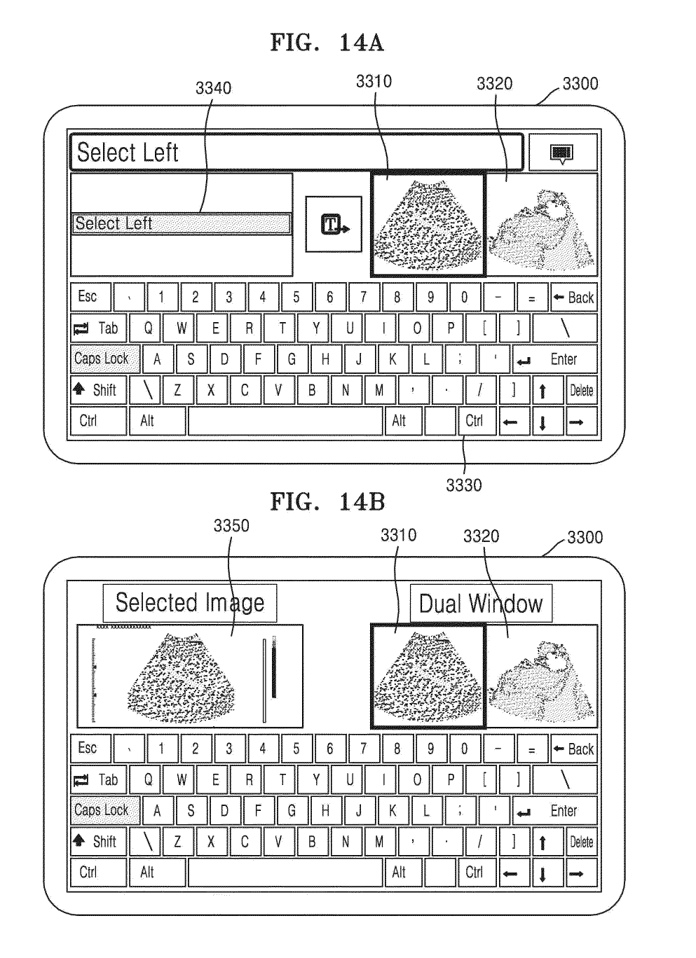

FIGS. 14A to 17B are diagrams for describing another example in which an input unit according to an exemplary embodiment outputs an image-processed result.

The input unit 1601 may receive a user input that requests a selection of one image 3310 from among medical images 3310 and 3320 displayed on a screen 3300, and the image processor 1201 may duplicate the image 3310 which is selected based on a user input. Here, selecting one image 3310 from among the medical images 3310 and 3320 may denote that the image processor 1201 further generates the same image as the selected medical image 3310, or may denote that the input unit 1601 displays one more image, which is the same as the selected medical image 3310, on the screen 3300.

Referring to FIG. 14A, a user may input data through a keyboard image 3330. For example, if the term which means a selection of a left image 3310 from among the medical images 3310 and 3320 is “select left”, the left image 3310 may be selected by the user inputting “s”, “e”, “I”, “e”, “c”, “t”, “I”, “e”, “f”, and “t” through the keyboard image 3330. Also, the user may select “select left” from a recommendation word list 3340 displayed on the screen 3300.

Referring to FIG. 14B, both the displayed images 3310 and 3320 and the selected image 3350 may be displayed on the screen 3300. Here, the newly displayed image 3350 and the displayed images 3310 and 3320 may be displayed in different regions of the screen 3300.

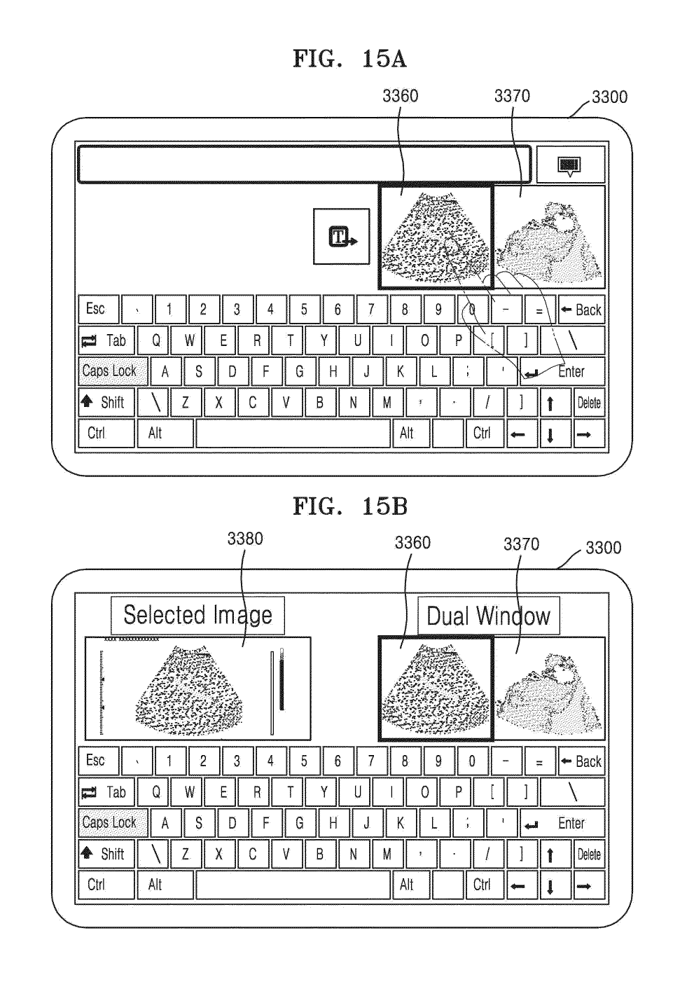

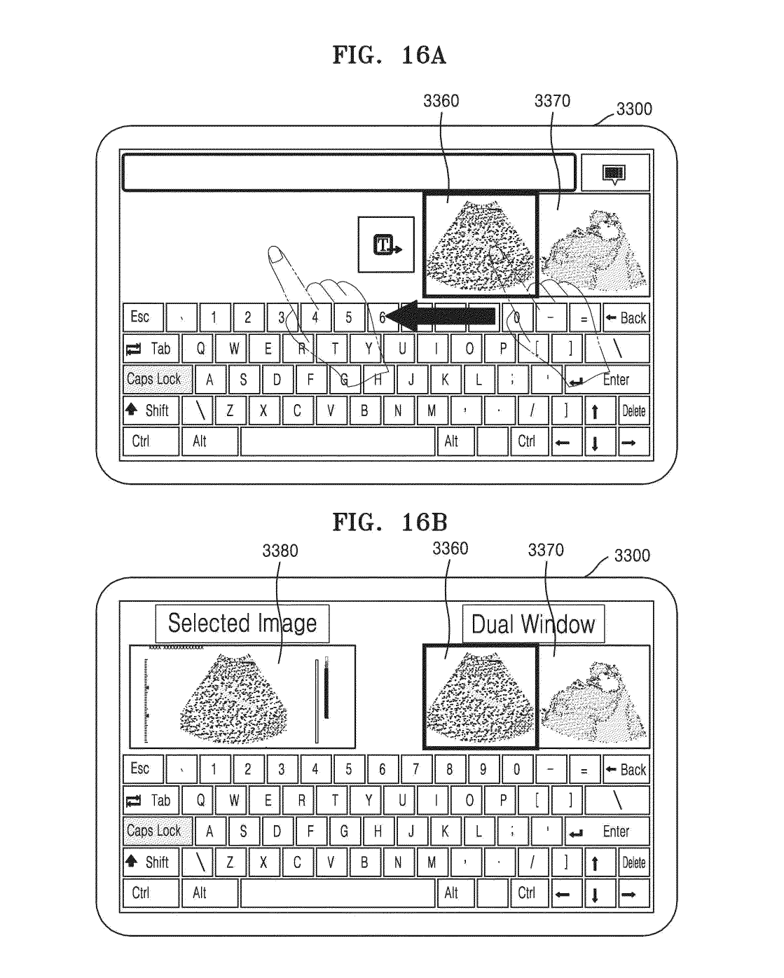

Referring to FIGS. 15A and 16A, the user may select one image 3360 from among displayed medical images 3360 and 3370 by applying a gesture to the screen 3300.

For example, referring to FIG. 15A, the medical image 3360 may be selected from among the displayed medical images 3360 and 3370 by the user tapping the medical image 3360. As another example, referring to FIG. 16A, the medical image 3360 may be selected from among the displayed medical images 3360 and 3370 by the user dragging and dropping the medical image 3360. A gesture made by the user is not limited to a tap or a drag and drop, and the medical image 3360 may be selected by the user making another predetermined gesture.

Referring to FIGS. 15B and 16B, both the displayed images 3360 and 3370 and a selected image 3380 may be displayed on the screen 3300. Here, the newly displayed image 3380 and the displayed images 3360 and 3370 may be displayed in different regions of the screen 3300.

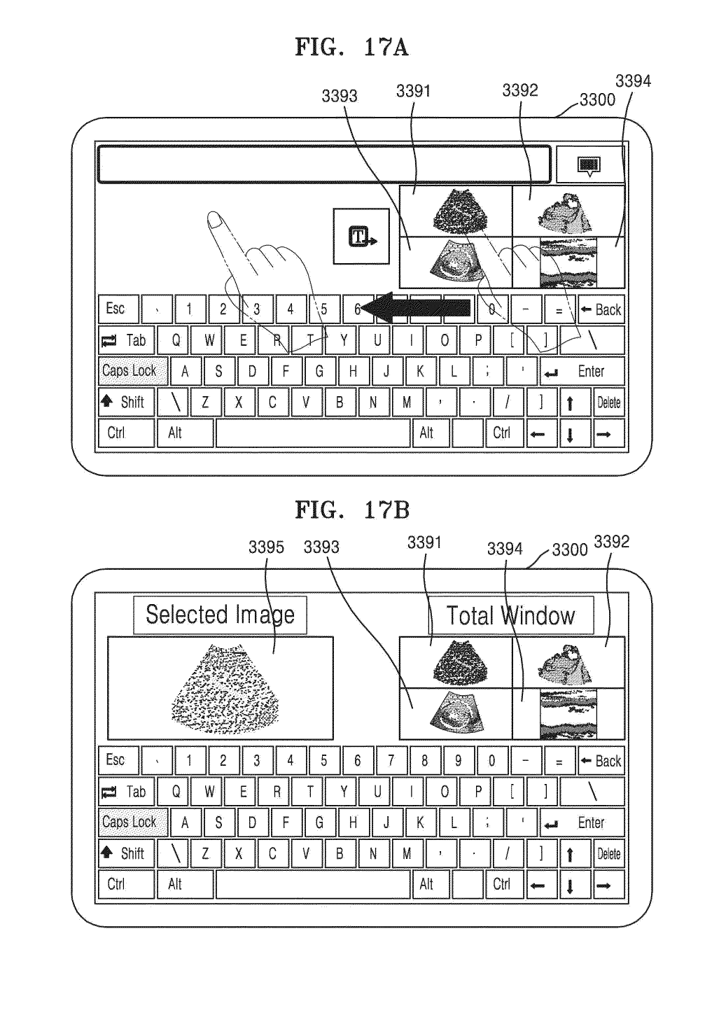

The number of medical images displayed on the screen 3300 is not limited to two, and a more number of images may be displayed. Referring to FIG. 17A, a total of four medical images 3391 to 3394 may be displayed on the screen 3300, and one image 3391 may be selected from among the four medical images 3391 to 3394 according to a user input. In this case, a process of selecting the medical image 3391 is as described above with reference to FIGS. 14A, 15A, and 16A. For example, one image 3391 may be selected from among the four medical images 3391 to 3394 by the user dragging and dropping the medical image 3391.

Referring to FIG. 17B, both the displayed medical images 3391 to 3394 and the selected image 3391 may be displayed on the screen 3300. Here, the newly displayed image 3391 and the displayed medical images 3391 to 3394 may be displayed in different regions of the screen 3300.

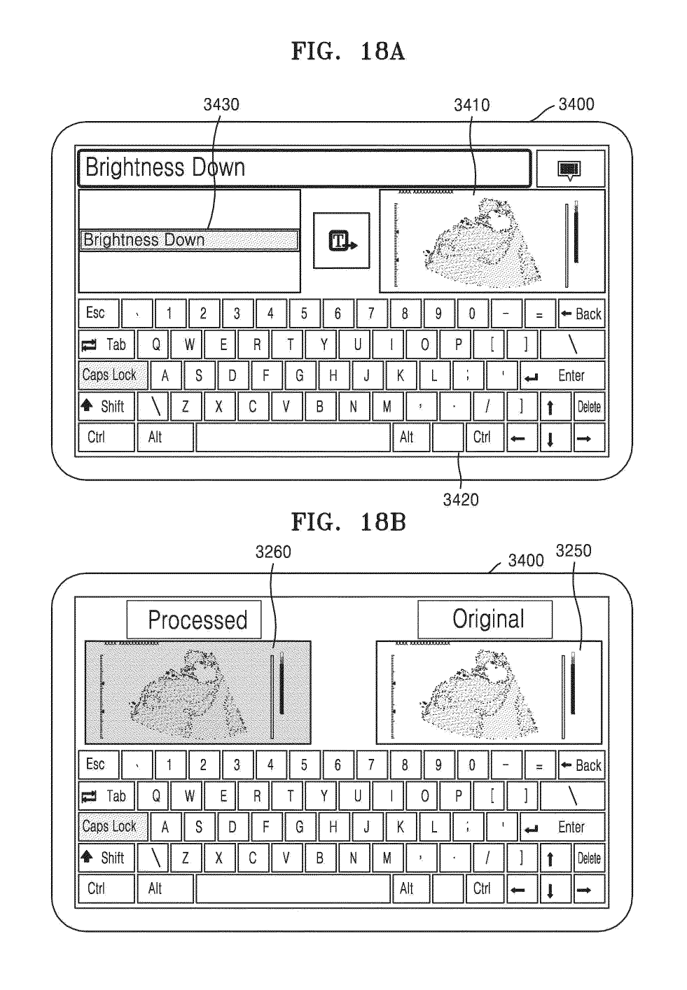

FIGS. 18A to 19B are diagrams for describing another example in which an input unit according to an exemplary embodiment outputs an image-processed result.

The input unit 1601 may receive a user input that requests changing of a brightness of a medical image 3410 displayed on a screen 3400, and the image processor 1201 may change a brightness of the medical image 3410, based on a user input. In other words, the image processor 1201 may generate a brighter image than a brightness of the medical image 3410. Hereinafter, only an example in which the image processor 1201 generates a darker image than a brightness of the medical image 3410 will be described, but an example in which the image processor 1201 generates a brighter image than a brightness of the medical image 3410 may be understood by one of ordinary skill in the art.

Referring to FIG. 18A, a user may input data through a keyboard image 3420. For example, if the term which means darkening of a brightness of the medical image 3410 is “brightness down”, the medical image 3410 may be duplicated by the user inputting “b”, “r”, “i”, “g”, “h”, “t”, “n”, “e”, “s”, “s”, “d”, “o”, “w”, and “n” through the keyboard image 3420. Also, the user may select “brightness down” from a recommendation word list 3430 displayed on the screen 3400.

Referring to FIG. 18B, both the displayed image 3410 and the brightness-darkened image 3440 may be displayed on the screen 3400. Here, the newly displayed image 3440 and the displayed image 3410 may be displayed in different regions of the screen 3400.

Referring to FIG. 19A, the user may adjust a brightness of a medical image 3450 by applying a gesture to the screen 3400. For example, a brightness of the medical image 3450 may be adjusted by the user dragging a certain region of the screen 3400. When the user drags the certain region, the input unit 1601 may display a brightness bar 3460 on the screen 3400, thereby informing the user that a brightness of the medical image 3450 is being adjusted. Also, the input unit 1601 may display (3470) a brightness degree of the medical image 3450 in a region adjacent to the brightness bar 3460.

In other words, the input unit 1601 may display the brightness bar 3460 on the screen 3400 while the user is making a drag gesture, and display (3470) a brightness degree of the medical image 3450 in correspondence with a position of the screen 3400 which is being dragged by the user. Therefore, the use easily recognizes how a brightness of the medical image 3450 is being changed.

Referring to FIG. 19B, both the displayed medical image 3450 and a brightness-adjusted image 3480 may be displayed on the screen 3400. Here, the newly displayed image 3480 and the displayed medical image 3450 may be displayed in different regions of the screen 3400.

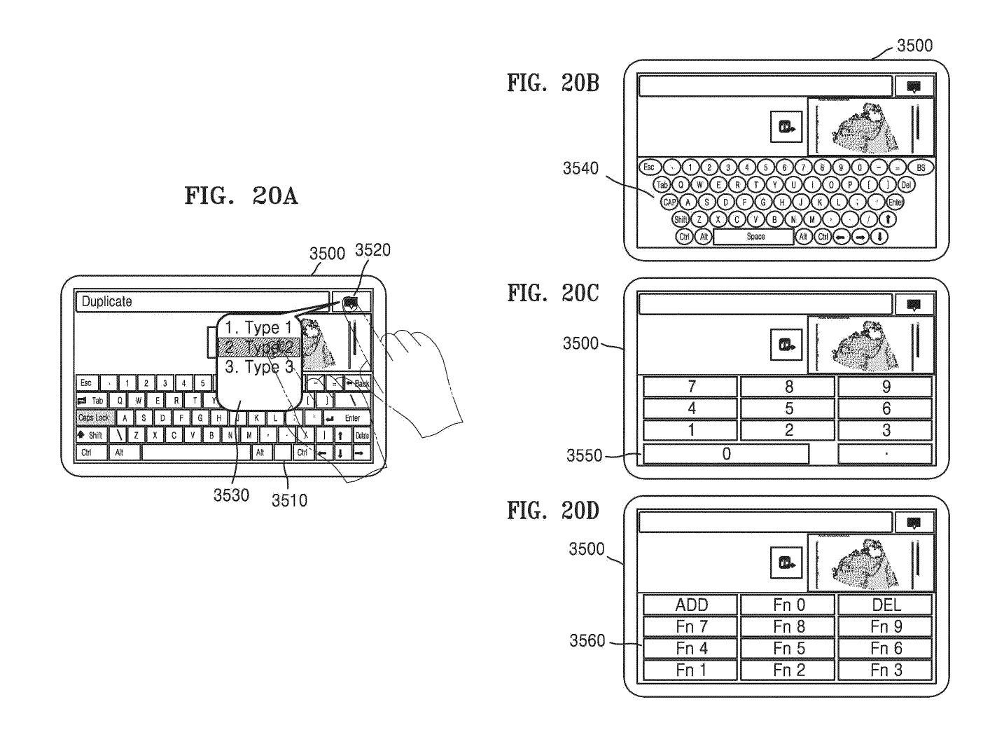

According to the details described above with reference to FIGS. 1 to 19B, the input unit 1601 may display a keyboard image, which is the same as a shape of a general keyboard, on a screen. The keyboard image is not limited to the above described, and various keyboard images based on a predetermined keyboard type may be displayed. Hereinafter, examples of a keyboard image will be described with reference to FIGS. 20A to 20D.

FIGS. 20A to 20D are diagrams for describing an example in which an input unit according to an exemplary embodiment outputs images of various types of keyboards.

Referring to FIG. 20A, a keyboard image 3510 which is the same as a shape of a general keyboard may be displayed on a screen 3500. In this case, when a user selects an icon 3520 displayed on the screen 3500, the input unit 1601 may display various types of keyboard images on the screen 3500 according to the selection of the user.

For example, when the user selects (for example, touch) the icon 3520, a plurality of predetermined keyboard types may be displayed in a popup window 3530. At this time, when the user selects one from among the predetermined keyboard types, the input unit 1601 may display a keyboard image, corresponding to the selected keyboard type, on the screen 3500.

Here, a keyboard type may be previously set through a separate setting operation performed by the user, or may be previously set by a manufacturer of the apparatus 101.

For example, referring to FIG. 20B, the input unit 1601 may display a keyboard image 3540, having a shape or a color which differs from that of the displayed keyboard image 3510. Here, the keyboard image 3540 having a shape or a color which differs from that of the keyboard image 3510 denotes that keys included in the keyboard image 3540 are the same as those of the keyboard image 3510, but a shape or a color of the keyboard image 3510 differs from a shape or a color of the keyboard image 3540.

As another example, referring to FIG. 20C, the input unit 1601 may display a keyboard image 3550, including keys which differ from the keys included in the displayed keyboard image 3510, on the screen 3500. For example, the keyboard image 3550 may be an image which includes only keys representing numbers. In addition, the keyboard image 3550 may be an image which includes only keys representing signs or special letters.

As another example, referring to FIG. 20D, the input unit 1601 may display a keyboard image 3560, which includes shortcut keys representing respective functions, on the screen 3500. For example, keys included in the keyboard image 3560 may be shortcut keys representing various methods (for example, add a text to an image, enlarge/reduce the image, adjust a brightness of the image, select one from among a plurality of images, and duplicate an image) in which the image processor 1201 processes a medical image. Therefore, when a user selects one key included in the keyboard image 3560, a function corresponding to the selected key may be executed even without inputting a separate command or gesture.

Types of a keyboard image are not limited to FIGS. 20B to 20D, and all types representing a keyboard image which differs from a keyboard image set as a default value may be applied without being limited.

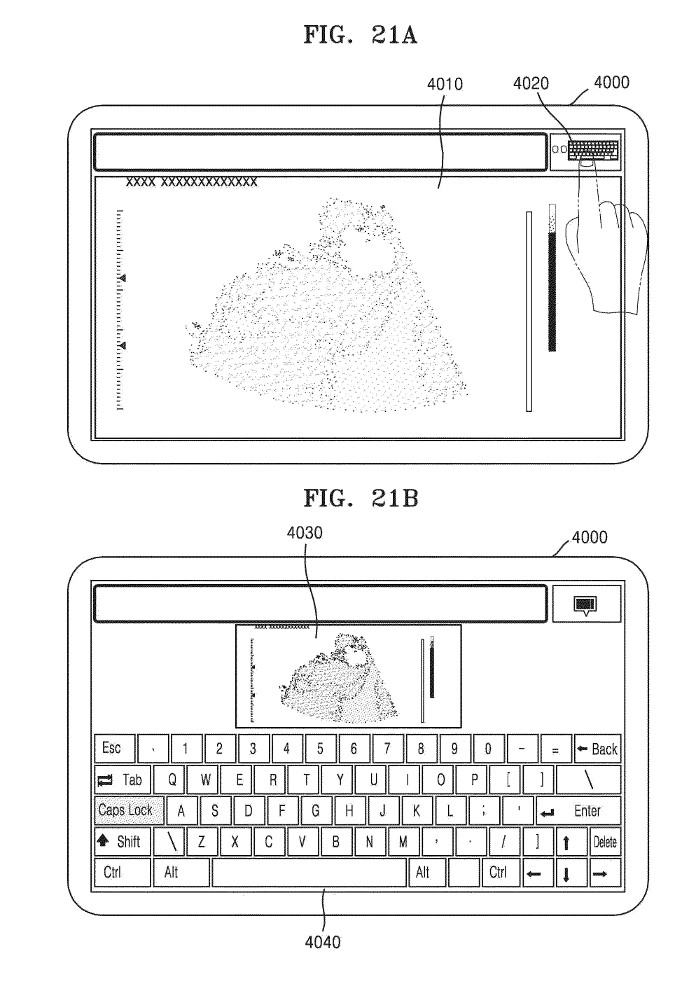

According to the details described above with reference to FIGS. 5 to 20D, both a medical image and a keyboard image are displayed on a screen of the input unit 1601. However, the input unit 1601 may first display the medical image on the screen, and display the keyboard image, based on a user input which is subsequently received. Alternatively, the input unit 1601 may first display the keyboard image on the screen, and display the medical image, based on a user input which is subsequently received. Also, the input unit 1601 may display the keyboard image and the medical image at a time, based on a user input. Hereinafter, this will be described in detail with reference to FIGS. 21 to 23.

FIGS. 21A to 23B are diagrams for describing a sequence in which a medical image and a keyboard image are output to an input unit according to an exemplary embodiment.

Referring to FIG. 21A, a medical image 4010 may be displayed on a screen 4000. In this state, a user may input a user input which requests an output of a keyboard image. For example, when the user selects an icon 4020 displayed in one region of the screen 4000, the input unit 1601 may additionally display the keyboard image on the screen 4000.

Referring to FIG. 21B, an example in which both a medical image 4030 and a keyboard image 4040 are displayed on the screen 4000 according to a user input is illustrated. For example, in a case where a region of the screen 4000 in which the keyboard image 4040 is to be displayed does not exist or is small due to a size of the displayed medical image 4010, the input unit 1601 may display the keyboard image 4040 on the screen 4000 and simultaneously display the medical image 4030 of which a size is reduced.

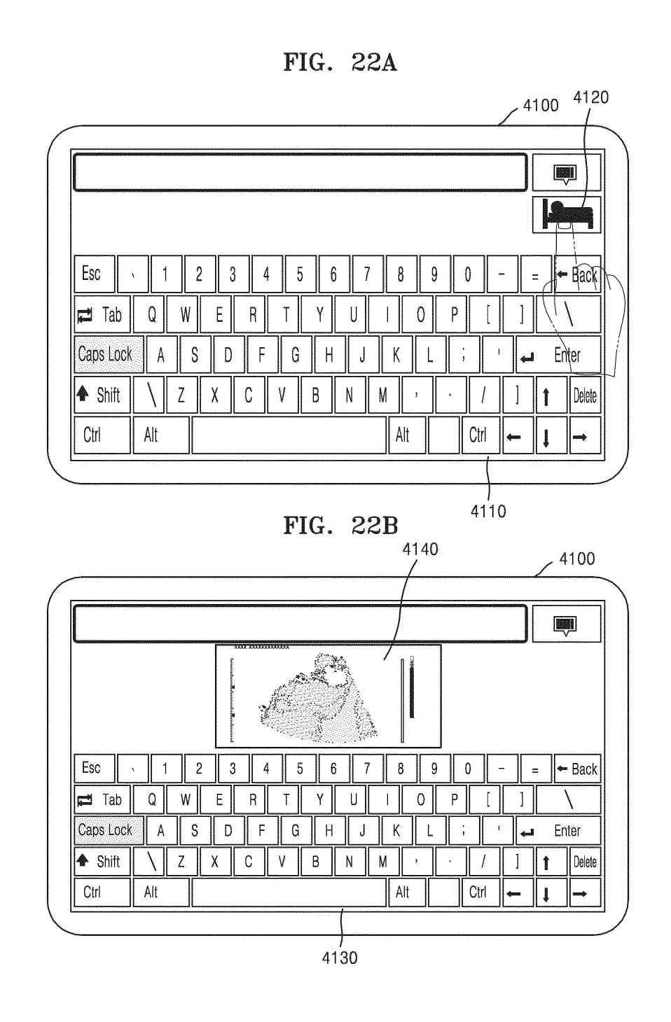

Referring to FIG. 22A, a keyboard image 4110 is displayed on a screen 4100. In this state, a user may input a user input which requests an output of a medical image. For example, when the user selects an icon 4120 displayed in one region of the screen 4100, the input unit 1601 may additionally display the medical image on the screen 4100.

Referring to FIG. 22B, an example in which both a medical image 4130 and a keyboard image 4140 are displayed on the screen 4100 according to a user input is illustrated. For example, in a case where a region of the screen 4100 in which the keyboard image 4140 is to be displayed does not exist or is small due to a size of the displayed keyboard image 4110, the input unit 1601 may display the keyboard image 4140 on the screen 4100 and simultaneously display the medical image 4130 of which a size is reduced.

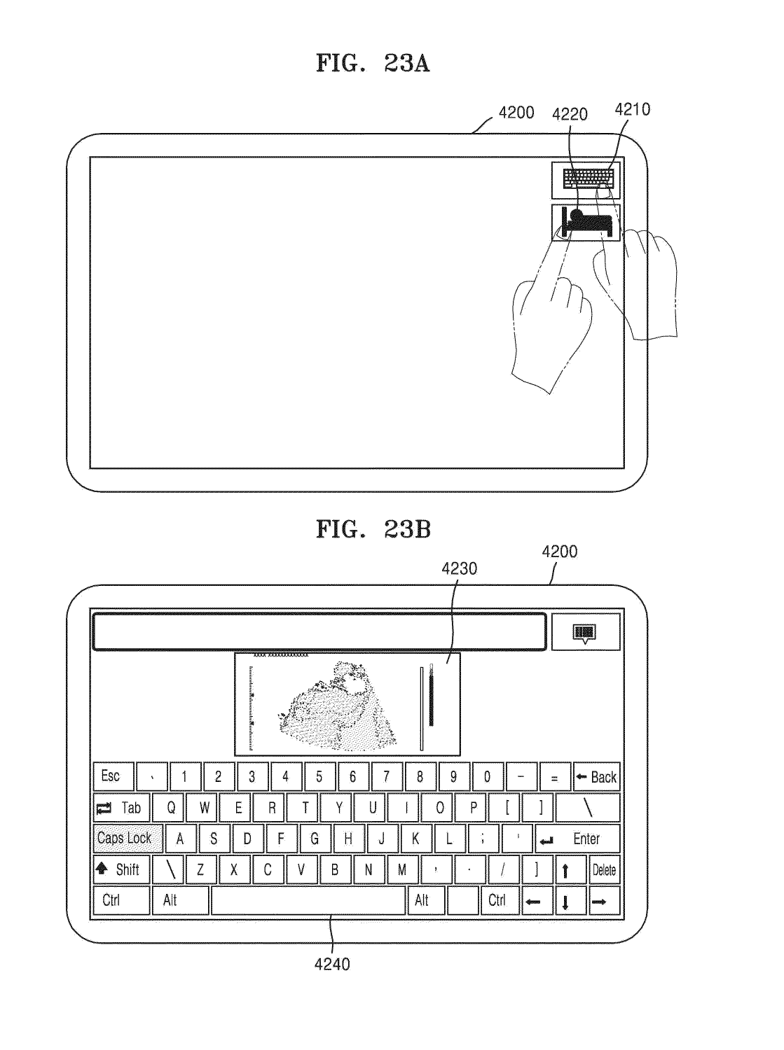

Referring to FIG. 23A, any image is not displayed on a screen 4200. In this state, a user may input a user input which requests an output of both a medical image and a keyboard image. For example, when the user simultaneously or continuously selects icons 4210 and 4220 displayed in one region of the screen 4200, as illustrated in FIG. 23B, the input unit 1601 may display both a medical image 4230 and a keyboard image 4240 on the screen 4200.

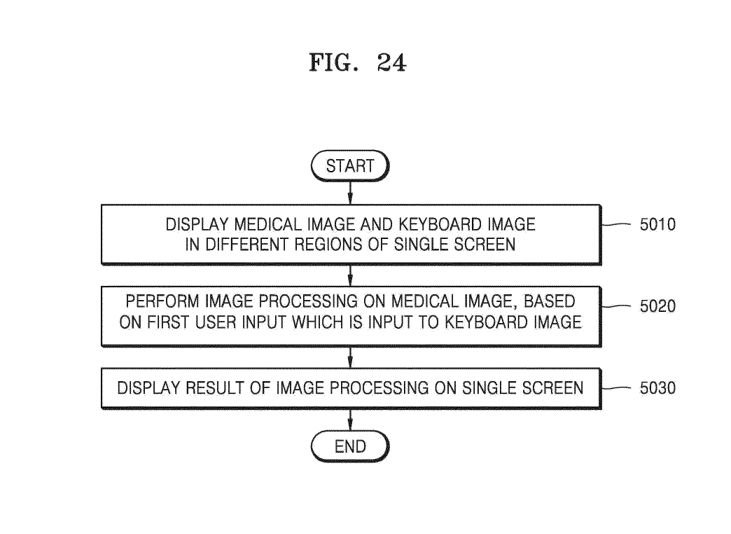

FIG. 24 is a flowchart for describing an example of a method of outputting a medical image and a keyboard image, according to an exemplary embodiment.

Referring to FIG. 24, a method of outputting a medical image and a keyboard image include operations which are time-serially performed by the ultrasound diagnosis systems 1000, 1001 and 1002 illustrated in FIGS. 1, 2 and 4 or the apparatuses 100 and 101. Therefore, it may be seen that among details which are not described below, details described above in the ultrasound diagnosis systems 1000, 1001 and 1002 illustrated in FIGS. 1, 2 and 4 or the apparatuses 100 and 101 are applied to the method of outputting a medical image and a keyboard image illustrated in FIG. 24.

In operation 5010, the input unit 1601 displays a medical image and a keyboard image in different regions of a single screen. For example, the medical image may be an ultrasound image which represents the object 10 (or an ROI of the object 10), but is not limited thereto. The medical image may include various kinds of images such as a magnetic resonance (MR) image, an X-ray image, a CT image, a PET image, and an OCT image, in addition to the ultrasound image.

The keyboard image may be an image which represent keys included in a general keyboard, but is not limited thereto. For example, the keyboard image may be an image which is generated based on a predetermined keyboard type. In this case, the keyboard type may be set by a user or may be set by a manufacturer or a seller of the apparatus 101.

The input unit 1601 receives a user input. Here, the user input may be input to the keyboard image. For example, when the user touches one or more keys in the keyboard image displayed by the input unit 1601, the input unit 1601 may receive a user input.

Moreover, the user input may be input to the medical image. For example, when a user applies a gesture to the medical image displayed by the input unit 1601, the input unit 1601 may receive the user input.

In operation 5020, the image processor 1201 performs image processing on the medical image, based on the user input. For example, the image processor 1201 may add a text to a portion of the medical image. As another example, the image processor 1201 may enlarge or reduce a certain region of the medical image. As another example, the image processor 1201 may change (or adjust) a brightness of the medical image. As another example, the image processor 1201 may duplicate a pre-generated medical image.

In operation 5030, the input unit 1601 outputs a result (i.e., an image for which image processing has been performed) of the image processing performed by the image processor 1201. At this time, the input unit 1601 may display both a before-image-processing image and an image-processing-performed image. Also, the input unit 1601 may display a plurality of images and an image which is selected from among the plurality of images by the user. Also, the input unit 1601 may display words, including a spelling which is selected according to a user input, along with the medical image and the keyboard image.

According to the above-described details, in a case where the user selects a desired key from a keyboard, an inconvenience of alternately looking at the medical image and the keyboard displayed by the display 1400 is avoided.

Moreover, the image processor may variously perform image processing on the medical image, based on a user input which is input through the keyboard image. Also, both a before-image-processing image and an after-image-processing image may be displayed on a screen of the input unit 1601, and thus, the user easily checks a result of image processing.

Moreover, the user may previously set a type of a keyboard image, and thus, the user may output and use an appropriate keyboard image depending on usability.

The above-described method may be written as computer programs and may be implemented in general-use digital computers that execute the programs using computer-readable recording media. A structure of data used in the above-described method may be recorded in computer-readable recording media through various members. Examples of the computer-readable recording medium include magnetic storage media (e.g., read-only memory (ROM), random access memory (RAM), universal serial bus (USB), floppy disks, and hard disks) and optical reading media (e.g., CD-ROMs and digital video disks (DVDs)).

It should be understood that the exemplary embodiments described herein should be considered in a descriptive sense only and not for purposes of limitation. Descriptions of features or aspects within each exemplary embodiment should typically be considered as available for other similar features or aspects in other exemplary embodiments.

While one or more exemplary embodiments have been described with reference to the figures, it will be understood by those of ordinary skill in the art that various changes in form and details may be made therein without departing from the spirit and scope as defined by the following claims.

What is claimed is:

1. A method of outputting a medical image representing an object and a keyboard image, the method comprising:
displaying the medical image and the keyboard image in different regions of a single screen;
performing image processing on the medical image, based on a first user input which is input via the keyboard image; and
displaying a result of the image processing on the single screen.

2. The method of claim 1, wherein the result of the image processing comprises an image in which a text is added to at least one portion of the medical image.

3. The method of claim 1, wherein the result of the image processing comprises an image which is obtained by enlarging a certain region of the medical image or an image which is obtained by reducing a certain region of the medical image.

4. The method of claim 1, wherein the result of the image processing comprises an image which is obtained by duplicating the medical image.

5. The method of claim 1, wherein the result of the image processing comprises an image which is obtained by changing a brightness of the medical image.

6. The method of claim 1, wherein the displaying of the result comprises simultaneously displaying the medical image and the result of the image processing on the single screen.

7. The method of claim 1, wherein the keyboard image comprises an image which is generated based on at least one keyboard type which is previously set.

8. The method of claim 1, wherein the displaying of the medical image and the keyboard image comprises displaying the keyboard image, based on a user input which is input while the medical image is displayed on the single screen.

9. The method of claim 1, wherein the displaying of the medical image and the keyboard image comprises displaying the medical image, based on a user input which is input while the keyboard image is displayed on the single screen.

10. The method of claim 1, further comprising performing image processing on the medical image, based on a second user input which is input via the medical image,
wherein the displaying of the result comprises displaying a result of the image processing, performed based on the second user input, on the single screen.

11. The method of claim 1, further comprising displaying at least one word, including at least one letter which is selected according to the first user input, on the single screen.

12. The method of claim 1, wherein the medical image comprises an image which is generated from a plurality of echo signals respectively corresponding to a plurality of ultrasound signals transmitted to the object.

13. A non-transitory computer-readable storage medium storing a program for executing the method of claim 1.

14. An apparatus for outputting a medical image representing an object and a keyboard image, the apparatus comprising:
an input unit that displays the medical image and the keyboard image in different regions of a single screen; and
an image processor that performs image processing on the medical image, based on a first user input which is input via the keyboard image,
wherein the input unit displays a result of the image processing on the single screen.

15. The apparatus of claim 14, wherein the result of the image processing comprises an image in which a text is added to at least one portion of the medical image.

16. The apparatus of claim 14, wherein the result of the image processing comprises an image which is obtained by enlarging a certain region of the medical image or an image which is obtained by reducing a certain region of the medical image.

17. The apparatus of claim 14, wherein the result of the image processing comprises an image which is obtained by duplicating the medical image.

18. The apparatus of claim 14, wherein the result of the image processing comprises an image which is obtained by changing a brightness of the medical image.

19. The apparatus of claim 14, wherein the input unit simultaneously displays the medical image and the result of the image processing on the single screen.

20. The apparatus of claim 14, wherein the keyboard image comprises an image which is generated based on at least one keyboard type which is previously set.

21. The apparatus of claim 14, wherein the input unit displays the keyboard image, based on a user input which is input while the medical image is displayed on the single screen.

22. The apparatus of claim 14, wherein the input unit displays the medical image, based on a user input which is input while the keyboard image is displayed on the single screen.

23. The apparatus of claim 14, wherein,
the image processor performs image processing on the medical image, based on a second user input which is input via the medical image, and
the input unit displays a result of the image processing, performed based on the second user input, on the single screen.

24. The apparatus of claim 14, wherein the input unit displays at least one word, including at least one letter which is selected according to the first user input, on the single screen.

25. The apparatus of claim 14, wherein the medical image comprises an image which is generated from a plurality of echo signals respectively corresponding to a plurality of ultrasound signals transmitted to the object.

26. An ultrasound diagnosis system for outputting a medical image representing an object and a keyboard image, the ultrasound diagnosis system comprising:
a probe that transmits a plurality of ultrasound signals to the object and receives a plurality of echo signals respectively corresponding to the plurality of ultrasound signals; and
an ultrasound imaging apparatus that generates the medical image by using the plurality of echo signals, displays the medical image and the keyboard image in different regions of a single screen, performs image processing on the medical image, based on a first user input which is input via the keyboard image, and displays a result of the image processing on the single screen.
